(19) United States
(12) Patent Application Publication
(10) Pub. No.: US 2009/0124806 A1
(43) Pub. Date: May 14, 2009

(54) PROCESS FOR PRODUCING CARBOXYLIC ACID FROM PRIMARY ALCOHOL

(75) Inventors: Yoshiharu IWABUCHI, Sendai-shi (JP); Masatoshi Shibuya, Sendai-shi (JP); Masaki Tomizawa, Sendai-shi (JP); Takahisa Sato, Sendai-shi (JP); Susumu Sato, Yokote-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Nissan Chemical Industries, Ltd., Chiyoda-ku (JP)

(21) Appl. No.: 12/115,847

(22) Filed: May 6, 2008

(30) Foreign Application Priority Data
Nov. 8, 2007 (JP) 2007-291108

(51) Int. Cl. C07D 471/08; C07D 211/94

(52) U.S. Cl. 546/72; 546/184

(57) ABSTRACT

To provide an industrially-useful and environmentally-friendly novel oxidation reaction.

A process for producing a carboxylic acid from a primary alcohol, which comprises using an alkali metal chlorite as a co-oxidizing agent and using, as a catalyst, an oxoammonium salt of the formula (1):

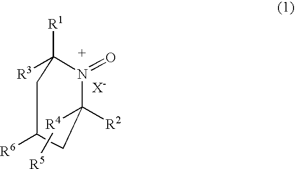

### BACKGROUND OF THE INVENTION

1. Field of the Invention

The present invention relates to a process for producing a carboxylic acid from a primary alcohol, wherein an oxoammonium salt catalyst is employed.

2. Discussion of Background

An oxidation reaction of a primary alcohol to a carboxylic acid is one of important reactions which are frequently used for the production of e.g. pharmaceutical or chemical products.

A method of employing a heavy metal salt oxidizing agent such as a chromate has been commonly used for a long time. In recent years, reflecting an environmental harmonization concept, a method of employing a hypervalent iodine reagent, or a catalytic method which makes it possible to utilize an environmentally-friendly co-oxidizing agent, has been developed (e.g. Non-Patent Documents 1 and 2).

Further, also known as an environmentally-friendly oxidation method is TEMPO oxidation employing a nitroxyl radical catalyst such as 2,2,6,6-tetramethylpiperidin-N-oxyl (hereinafter referred to as TEMPO) and, as a bulk oxidizing agent, an aqueous sodium hypochlorite solution or iodobenzene diacetate (e.g. Non-Patent Documents 3 and 4), or oxidation employing 1-Me-AZADO (e.g. Patent Document 2).

As an oxidation method employing a chlorite as a bulk oxidizing agent, a method has been developed wherein a primary alcohol is oxidized to an aldehyde by TEMPO oxidation, and then the aldehyde is oxidized to a carboxylic acid by the chlorite present in the system (e.g. Patent Document 1, Non-Patent Documents 5 and 6).

As a one-pot oxidation reaction, a method is known wherein firstly, a primary alcohol is oxidized to an aldehyde by an aqueous sodium hypochlorite solution in a weakly basic condition, and after adjusting the system to a weakly acidic condition with hydrochloric acid, the aldehyde is oxidized to a carboxylic acid by sodium chlorite (e.g. Non-Patent Document 7).

Patent Document 1: WO99/52849

Patent Document 2: WO2006/001387

Non-Patent Document 1: Noyori R. et al., Chem. Commun. 2003. 1977

Non-Patent Document 2: Kita Y. et al., Angew. Chem. Int. Ed. 2000, vol. 39, p1306

Non-Patent Document 3: Anelli, P. L. et al., J. Org. Chem., 1987, vol. 52, p2559

Non-Patent Document 4: Theodore S. Widlanski, et al., J. Org. Chem., 1999, vol. 64, p293

Non-Patent Document 5: Mangzhu Zhao et al., J. Org. Chem., 1999, vol. 64, p2564

Non-Patent Document 6: Andreas Kirschning et al., Adv. Synth. Catal., 2005, vol. 347, p1423

Non-Patent Document 7: Atsuhiko Zenka, Chem. Pharm. Bull., 2003, vol. 51, p888

Each of such conventional methods still has a problem from the viewpoint of substrate adaptability, and it is desired to develop a practical oxidation reaction which has both wide substrate adaptability and environmental harmonization property. This is evident also from the fact that in many cases, a two step method is still employed wherein by oxidation of a primary alcohol, an aldehyde is once isolated, and then by a combined use of Pinnick oxidation employing sodium chlorite, a carboxylic acid is obtained. The method disclosed in Patent Document 1 and Non-Patent Document 5 is relatively better in the substrate adaptability, but it requires a heating condition at a temperature of from 35 to 50° C. and requires a strict reaction control such that the respective aqueous solutions of sodium hypochlorite and sodium chlorite, are dropwise added simultaneously over a long time. Thus, further improvement for practical utility has been desired.

### SUMMARY OF THE INVENTION

It is an object of the present invention to improve TEMPO oxidation using a catalytic amount of a nitroxyl radical and a bulk oxidizing agent, which has been used in recent years as a practically useful method to some extent and to provide a simple and efficient oxidation reaction of a primary alcohol to a carboxylic acid, which has both wide substrate adaptability and environmental harmonization property.

The present inventors have conducted an extensive study to accomplish the above object and as a result, have accomplished the present invention wherein an oxoammonium salt is used as a catalyst, and an alkali metal hypochlorite is used as a bulk oxidizing agent.

Namely, the present invention provides the following:

(1) A process for producing a carboxylic acid from a primary alcohol, which comprises using an alkali metal chlorite as a co-oxidizing agent and using, as a catalyst, an oxoammonium salt of the formula (1):

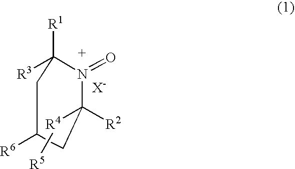

wherein X⁻ is a counter ion, each of R¹, R², R³, R⁴ and R⁵ which are independent of one another, is at least one substituent selected from a hydrogen atom, a halogen atom, a nitro group, a cyano group, a hydroxyl group, a mercapto group, an amino group, a formyl group, a carboxyl group, a sulfo group, a linear or branched C₁₋₁₂ alkyl group, a C₃₋₁₂ cycloalkyl group, a (linear or branched C₁₋₁₂ alkyl)oxy group, a (C₃₋₁₂ cycloalkyl)oxy group, a (linear or branched C₁₋₁₂ alkyl)thio group, a (C₃₋₁₂ cycloalkyl)thio group, a (linear or branched C₁₋₁₂ alkyl)amino group, a (C₃₋₁₂ cycloalkyl)amino group, a di(linear or branched C₁₋₆ alkyl)amino group, a di(C₃₋₆ cycloalkyl)amino group, a linear or branched C₁₋₁₂ alkylcarbonyl group, a C₃₋₁₂ cycloalkylcarbonyl group, a (linear or branched C₁₋₁₂ alkyl)oxycarbonyl group, a (C₃₋₁₂ cycloalkyl)oxycarbonyl group, a (linear or branched C₁₋₁₂ alkyl)thiocarbonyl group, a (C₃₋₁₂ cycloalkyl)thiocarbonyl group, a (linear or branched C₁₋₁₂ alkyl)aminocarbonyl group, a (C₃₋₁₂ cycloalkyl)aminocarbonyl group, a di(linear or branched C₁₋₆ alkyl)aminocarbonyl group, a di(C₃₋₆ cycloalkyl)aminocarbonyl group, a (linear or branched C₁₋₁₂ alkyl)carbonyloxy group, a (C₃₋₁₂ cycloalkyl)carbonyloxy group, a (linear or branched C₁₋₁₂ alkyl)carbonylthio group, a (C₃₋₁₂ cycloalkyl)carbonylthio group, a (linear or branched C₁₋₁₂ alkyl)carbonylamino group, a (C₃₋₁₂ cycloalkyl)carbonylamino group, a di(linear or branched C₁₋₁₂ alkylcarbonyl)amino group, a di(C₃₋₁₂ cycloalkylcarbonyl)amino group, a linear or branched C₁₋₆ haloalkyl group, a C₃₋₆ halocycloalkyl group, a linear or branched C₂₋₆ alkenyl group, a C₃₋₆ cycloalkenyl group, a linear or branched C₂₋₆ haloalkenyl group, a C₃₋₆ halocycloalkenyl group, a linear or branched C₂₋₆ alkynyl group, a linear or branched C₂₋₆ haloalkynyl group, a benzyl group which may be substituted by R^{a}, a benzyloxy group which may be substituted by Rₐ, a benzylthio group which may be substituted by R^{a}, a benzylamino group which may be substituted by R^{a}, a benzylcarbonyl group which may be substituted by R^{a}, a benzyloxycarbonyl group which may be substituted by R^{a}, a benzylthiocarbonyl group which may be substituted by R^{a}, a benzylaminocarbonyl group which may be substituted by R^{a}, a dibenzylaminocarbonyl group which may be substituted by R^{a}, a benzylcarbonyloxy group which may be substituted by R^{a}, a benzylcarbonylthio group which may be substituted by R^{a}, a benzylcarbonylamino group which may be substituted by R^{a}, a di(benzylcarbonyl)amino group which may be substituted by R^{a}, an aryl group which may be substituted by R^{a}, an aryloxy group which may be substituted by R^{a}, an arylthio group which may be substituted by R^{a}, an arylamino group which may be substituted by R^{a}, a diarylamino group which may be substituted by R^{a}, an arylcarbonyl group which may be substituted by R^{a}, an aryloxycarbonyl group which may be substituted by R^{a}, an arylthiocarbonyl group which may be substituted by R^{a}, an arylaminocarbonyl group which may be substituted by R^{a}, a diarylaminocarbonyl group which may be substituted by R^{a}, an arylcarbonyloxy group which may be substituted by R^{a}, an arylcarbonylthio group which may be substituted by R^{a}, an arylcarbonylamino group which may be substituted by R^{a}, and a di(arylcarbonyl)amino group which may be substituted by R^{a}, provided that when the number of substituents is 2 or more, the respective substituents may be the same or different, R³ and R⁴ may together represent CH₂CHR⁷CH₂, provided that each hydrogen atom in CH₂CHR⁷CH₂ may be substituted by R⁵, each of R⁶ and R⁷ which are independent of each other, has the same meaning as R⁵, or R⁶ and R⁷ may together form methylene which may be substituted by R⁵, R^{a} is halogen, a C₁₋₆ alkyl group, a C₁₋₆ haloalkyl group, a C₃₋₆ cycloalkyl group, a C₁₋₆ alkoxy group, a C₁₋₆ alkoxy C₁₋₆ alkyl group, a C₁₋₆ alkylsulfenyl C₁₋₆ alkyl group, a C₁₋₆ haloalkoxy group, a C₁₋₆ alkylsulfenyl group, a C₁₋₆ alkylsulfinyl group, a C₁₋₆ alkylsulfonyl group, a C₁₋₆ haloalkylsulfenyl group, a C₁₋₆ haloalkylsulfinyl group, a C₁₋₆ haloalkylsulfonyl group, a C₂₋₆ alkenyl group, a C₂₋₆ haloalkenyl group, a C₂₋₆ alkenyloxy group, a C₂₋₆ haloalkenyloxy group, a C₂₋₆ alkenylsulfenyl group, a C₂₋₆ alkenylsulfinyl group, a C₂₋₆ alkenylsulfonyl group, a C₂₋₆ haloalkenylsulfenyl group, a C₂₋₆ haloalkenylsulfinyl group, a C₂₋₆ haloalkenylsulfonyl group, a C₂₋₆ alkynyl group, a C₂₋₆ haloalkynyl group, a C₂₋₆ alkynyloxy group, a C₂₋₆ haloalkynyloxy group, a C₂₋₆ alkynylsulfenyl group, a C₂₋₆ alkynylsulfinyl group, a C₂₋₆ alkynylsulfonyl group, a C₂₋₆ haloalkynylsulfenyl group, a C₂₋₆ haloalkynylsulfinyl group, a C₂₋₆ haloalkynylsulfonyl group, a nitro group, a cyano group, a hydroxyl group, a mercapto group, an amino group, a formyl group, a carboxyl group, a sulfo group, a C₁₋₆ alkoxycarbonyl group, a C₁₋₆ alkylcarbonyl group, a C₁₋₆ haloalkylcarbonyl group, a C₁₋₆ alkylcarbonyloxy group, a phenyl group, a C₁₋₆ alkylamino group or a di-C₁₋₆ alkylamino group, provided that the number of R^{a} is from 1 to 5, and when the number of R^{a} is 2 or more, the respective substituents may be the same or different.

(2) The process according to (1), wherein in the formula (1), R³ and R⁴ together represent CH₂CHR⁷CH₂, provided that each hydrogen atom in CH₂CHR⁷CH₂ may be substituted by R⁵, and R⁶ and R⁷ together form methylene which may be substituted by R⁵.

(3) A process for producing an oxoammonium salt of the formula (3):

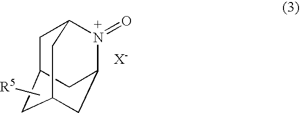

wherein R⁵ and X are as defined above, which comprises reacting halogen with a nitroxyl radical of the formula (2):

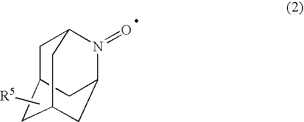

wherein R⁵ is as defined above.

(4) The process according to (1), wherein the counter ion is F⁻, Cl⁻, Br⁻ or I⁻.

(5) The process according to (2), wherein the counter ion is F⁻, Cl⁻, Br⁻ or I⁻.

(6) The process according to (3), wherein the counter ion is F⁻, Cl⁻, Br⁻ or I⁻.

According to the present invention, it becomes possible to simply and efficiently produce a desired carboxylic acid not only from a primary alcohol having a relatively simple structure but also from a primary alcohol having various functional groups such as unsaturated bonds or electron-rich aromatic rings, is without being chlorinated. The oxidation method of the present invention is not only useful for an experiment on a laboratory scale but also expected to contribute substantially to industrial production of pharmaceutical or chemical products.

### DETAILED DESCRIPTION OF THE PREFERRED EMBODIMENTS

In this specification, a halogen atom includes a fluorine atom, a chlorine atom, a bromine atom and an iodine atom. Further, in this specification, “halo” also represents such halogen atoms.

In this specification, “C_{a-b} alkyl” represents a linear or branched hydrocarbon group having from a to b carbon atoms. Specifically, it may, for example, be a methyl group, an ethyl group, a n-propyl group, an i-propyl group, a n-butyl group, an i-butyl group, a s-butyl group, a t-butyl group, a n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a 2-hexyl group, a 1 methylpentyl group, a 2-methylpentyl group, a 1,1-dimethylbutyl group, a 1,3-dimethylbutyl group, a heptyl group, an octyl group, a nonyl group, a decyl group, an undecyl group or a dodecyl group, and it is selected within a range of the specified number of carbon atoms.

In this specification, “Cab haloalkyl” represents a linear or branched hydrocarbon group having from a to b carbon atoms wherein hydrogen atoms bonded to carbon atoms are optionally substituted by halogen atoms, and when the hydrogen atoms are substituted by two or more halogen atoms, such halogen atoms may be the same or different from one another. Specifically, it may, for example, be a fluoromethyl group, a chloromethyl group, a bromomethyl group, an iodomethyl group, a difluoromethyl group, a chlorofluoromethyl group, a dichloromethyl group, a bromofluoromethyl group, a trifluoromethyl group, a chlorodifluoromethyl group, a dichlorofluoromethyl group, a trichloromethyl group, a bromodifluoromethyl group, a bromochlorofluoromethyl group, a dibromofluoromethyl group, a 2-fluoroethyl group, a 2-chloroethyl group, a 2-bromoethyl group, a 2,2-difluoroethyl group, a 2-chloro-2-fluoroethyl group, a 2,2-dichloroethyl group, a 2-bromo-2-fluoroethyl group, a 2,2,2-trifluoroethyl group, a 2-chloro-2,2-difluoroethyl group, a 2,2-dichloro-2-fluoroethyl group, a 2,2,2-trichloroethyl group, a 2-bromo-2,2-difluoroethyl group, a 2-bromo-2-chloro-2-fluoroethyl group, a 2-bromo-2,2-dichloroethyl group, a 1,1,2,2-tetrafluoroethyl group, a pentafluoroethyl group, a 1-chloro-1,2,2,2-tetrafluoroethyl group, a 2-chloro-1,1,2,2-tetrafluoroethyl group, a 1,2-dichloro-1,2,2-trifluoroethyl group, a 2-bromo-1,1,2,2-tetrafluoroethyl group, a 2-fluoropropyl group, a 2-chloropropyl group, a 2-bromopropyl group, a 2-chloro-2-fluoropropyl group, a 2,3-dichloropropyl group, a 2-bromo-3-fluoropropyl group, a 3-bromo-2-chloropropyl group, a 2,3-dibromopropyl group, a 3,3,3-trifluoropropyl group, a 3-bromo-3,3-difluoropropyl group, a 2,2,3,3-tetrafluoropropyl group, a 2-chloro-3,3,3-trifluoropropyl group, a 2,2,3,3,3-pentafluoropropyl group, a 1,1,2,3,3,3-hexafluoropropyl group, a heptafluoropropyl group, a 2,3-dichloro-1,1,2,3,3-pentafluoropropyl group, a 2-fluoro-1-methylethyl group, a 2-chloro-1-methylethyl group, a 2-bromo-1-methylethyl group, a 2,2,2-trifluoro-1-(trifluoromethyl)ethyl group, a 1,2,2,2-tetrafluoro-1-(trifluoromethyl)ethyl group, a 2,2,3,3,4,4-hexafluorobutyl group, a 2,2,3,4,4,4-hexafluorobutyl group, a 2,2,3,3,4,4,4-heptafluorobutyl group, a 1,1,2,2,3,3,4,4-octafluorobutyl group, a nonafluorobutyl group, a 4-chloro-1,1,2,2,3,3,4,4-octafluorobutyl group, a 2-fluoro-2-methylpropyl group, a 2-chloro-1,1-dimethylethyl group, a 2-bromo-1,1-dimethylethyl group, a 5-chloro-2,2,3,4,4,5,5-heptafluoropentyl group or a tridecafluorohexyl group, and it is selected within a range of the specified number of carbon atoms.

In this specification, “C_{a-b} cycloalkyl” represents a cyclic hydrocarbon group having from a to b carbon atoms, and it may form a 3- to 6-membered monocyclic or polycyclic structure. Further, each ring may optionally be substituted by an alkyl group within a range of the specified number of carbon atoms. Specifically, it may, for example, a cyclopropyl group, a 1-methylcyclopropyl group, a 2-methylcyclopropyl group, a 2,2-dimethylcyclopropyl group, a 2,2,3,3-tetramethylcyclopropyl group, a cyclobutyl group, a cyclopentyl group, a 2-methylcyclopentyl group, a 3-methylcyclopentyl group, a cyclohexyl group, a 2-methylcyclohexyl group, a 3-methylcyclohexyl group, a 4-methylcyclohexyl group or a bicyclo[2.2.1]heptan-2-yl group, and it is selected within a range of the specified number of carbon atoms.

In this specification, “C_{a-b} halocycloalkyl” represents a cyclic hydrocarbon group having from a to b carbon atoms, wherein hydrogen atoms bonded to carbon atoms are optionally substituted by halogen atoms, and it may form a 3- to 6-membered monocyclic or polycyclic structure. Further, each ring may optionally be substituted by an alkyl group within a range of the specified number of carbon atoms, and substitution by halogen atoms may be at the cyclic structure portion or at a side chain portion, or at both of them. Further, when the hydrogen atoms are substituted by two or more halogen atoms, such halogen atoms may be the same or different from one another. Specifically, it may, for example, be a 2,2-difluorocyclopropyl group, a 2,2-dichlorocyclopropyl group, a 2,2-dibromocyclopropyl group, a 2,2-difluoro-1-methylcyclopropyl group, a 2,2-dichloro-1-methylcyclopropyl group, a 2,2-dibromo-1-methylcyclopropyl group, 2,2,3,3-tetrafluorocyclobutyl group, a 2-(trifluoromethyl)cyclohexyl group, a 3-(trifluoromethyl)cyclohexyl group or a 4-(trifluoromethyl)cyclohexyl group, and it is selected within a range of the specified number of carbon atoms.

In this specification, “C_{a-b} alkenyl” represents a linear or branched unsaturated hydrocarbon group having from a to b carbon atoms and having one or more double bonds in its molecule. Specifically, it may, for example, be a vinyl group, a 1-propenyl group, a 2-propenyl group, a 1-methylethenyl group, a 2-butenyl group, a 1-methyl-2-propenyl group, a 2-methyl-2-propenyl group, a 2-pentenyl group, a 2-methyl-2-butenyl group, a 3-methyl-2-butenyl group, a 2-ethyl-2-propenyl group, a 1,1-dimethyl-2-propenyl group, a 2-hexenyl group, a 2-methyl-2-pentenyl group, a 2,4-dimethyl-2,6-heptadienyl group or a 3,7-dimethyl-2,6-octadienyl group, and it is selected within a range of the specified number of carbon atoms.

In this specification, “C_{a-b} haloalkenyl” represents a linear or branched unsaturated hydrocarbon group having from a to b carbon atoms and having one or more double bonds in its molecule, wherein hydrogen atoms bonded to carbon atoms are optionally substituted by halogen atoms. Here, when the hydrogen atoms are substituted by two or more halogen atoms, such halogen atoms may be the same or different from one another. Specifically, it may, for example, be a 2,2-dichlorovinyl group, a 2-fluoro-2-propenyl group, a 2-chloro-2-propenyl group, a 3-chloro-2-propenyl group, a 2-bromo-2-propenyl group, a 3-bromo-2-propenyl group, a 3,3-difluoro-2-propenyl group, a 2,3-dichloro-2-propenyl group, a 3,3-dichloro-2-propenyl group, a 2,3-dibromo-2-propenyl group, a 2,3,3-trifluoro-2-propenyl group, a 2,3,3-trichloro-2-propenyl group, a 1-(trifluoromethyl)ethenyl group, a 3-chloro-2-butenyl group, a 3-bromo-2-butenyl group, a 4,4-difluoro-3-butenyl group, a 3,4,4-trifluoro-3-butenyl group, a 3-chloro-4,4,4-trifluoro-2-butenyl group or a 3-bromo-2-methyl-2-propenyl group, and it is selected within a range of the specified number of carbon atoms.

In this specification, “C_{a-b} cycloalkenyl” represents a cyclic unsaturated hydrocarbon group having from a to b carbon atoms and having one or more double bonds, and it may form a monocyclic or polycyclic structure. Further, each ring may optionally be substituted by an alkyl group within a range of the specified number of carbon atoms. Further, the double bonds may be of an endo-type or an exo-type. Specifically, it may, for example, be a 2-cyclopenten-1-yl group, a 3-cyclopenten-1-yl group, a 2-cyclohexen-1-yl group, a 3-cyclohexen-1-yl group or a bicyclo[2.2.1]-5-hepten-2-yl group, and it is selected within a range of the specified number of carbon atoms.

In this specification, “C_{a-b} halocycloalkenyl” represents a cyclic unsaturated hydrocarbon group having from a to b carbon atoms and having one or more double bonds, wherein hydrogen atoms bonded to carbon atoms are optionally substituted by halogen atoms, and it may form a monocyclic or polycyclic structure. Further, each ring may optionally be substituted by an alkyl group within a range of the specified number of carbon atoms. Further, the double bonds may be of an endo-type or an exo-type. Further, substitution by a halogen atom may be at a cyclic structure portion or at a side chain portion, or at both of them, and when the hydrogen atoms are substituted by two or more halogen atoms, such halogen atoms may be the same or different from one another. Specifically, it may, for example, be a 2-chlorobicyclo[2.2.1]-5-hepten-2-yl group, and it is selected within a range of the specified number of carbon atoms.

In this specification, “C_{a-b} alkynyl” represents a linear or branched unsaturated hydrocarbon group having from a to b carbon atoms and having one or more triple bonds in its molecular weight. Specifically, it may, for example, be an ethynyl group, a 1-propynyl group, a 2-propynyl group, a 2-butynyl group, a 1-methyl-2-propynyl group, a 2-pentynyl group, a 1-methyl-2-butynyl group, a 1,1-dimethyl-2-propynyl group or a 2-hexynyl group, and it is selected within a range of the specified number of carbon atoms.

In this specification, “C_{a-b} haloalkynyl” represents a linear or branched unsaturated hydrocarbon group having from a to b carbon atoms and having one or more triple bonds in its molecule, wherein hydrogen atoms bonded to carbon atoms are optionally substituted by halogen atoms. Here, when the hydrogen atoms are substituted by two or more halogen atoms, such halogen atoms may be the same or different from one another. Specifically, it may, for example, be a 2-chloroethynyl group, a 2-bromoethynyl group, a 2-iodoethynyl group, a 3-chloro-2-propynyl group, a 3-bromo-2-propynyl group or a 3-iodo-2-propynyl group, and it is selected within a range of the specified number of carbon atoms.

The aryl group which may be substituted by R^{a} may, for example, be a phenyl group, an o-methylphenyl group, a m-methylphenyl group, a p-methylphenyl group, an o-chlorophenyl group, a m-chlorophenyl group, a p-chlorophenyl group, an o-fluorophenyl group, a p-fluorophenyl group, an o-methoxyphenyl group, a p-methoxyphenyl group, a p-nitrophenyl group, a p-cyanophenyl group, an α-naphthyl group, a β-naphthyl group, an o-biphenylyl group, a m-biphenylyl group, a p-biphenylyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 2-thienyl group, a 3-thienyl group, a 2-furyl group, a 3-furyl group, a 2-pyranyl group, a 3-pyranyl group, a 4-pyranyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 4-isobenzofuranyl group, a 5-isobenzofuranyl group, a 2-benzothienyl group, a 3-benzothienyl group, a 4-benzothienyl group, a 5-benzothienyl group, a 6-benzothienyl group, a 7-benzothienyl group, a 1-isobenzothienyl group, a 4-isobenzothienyl group, a 5-isobenzothienyl group, a 2-chromenyl group, a 3-chromenyl group, a 4-chromenyl group, a 5-chromenyl group, a 6-chromenyl group, a 7-chromenyl group, a 8-chromenyl group, a 1-pyrrolyl group, a 2-pyrrolyl group, a 3-pyrrolyl group, a 1-imidazolyl group, a 2-imidazolyl group, a 4-imidazolyl group, a 1-pyrazolyl group, a 3-pyrazolyl group, a 4-pyrazolyl group, a 2-thiazolyl group, a 4-thiazolyl group, a 5-thiazolyl group, a 3-isothiazolyl group, a 4-isothiazolyl group, a 5-isothiazolyl group, a 2-oxazolyl group, a 4-oxazolyl group, a 5-oxazolyl group, a 3-isoxazolyl group, a 4-isoxazolyl group, a 5-isoxazolyl group, a 2-pyridyl group, a 3-pyridyl group, a 4-pyridyl group, a 2-pyrazinyl group, a 2-pyrimidinyl group, a 4-pyrimidinyl group, a 5-pyrimidinyl group, a 3-pyridazinyl group, a 4-pyridazinyl group, a 1-indolizinyl group, a 2-indolizinyl group, a 3-indolizinyl group, a 5-indolizinyl group, a 6-indolizinyl group, a 7-indolizinyl group, a 8-indolizinyl group, a 1-isoindryl group, a 4-isoindryl group, a 5-isoindryl group, a 1-indryl group, a 2-indryl group, a 3-indryl group, a 4-indryl group, a 5-indryl group, a 6-indryl group, a 7-indryl group, a 1-indazolyl group, a 2-indazolyl group, a 3-indazolyl group, a 4-indazolyl group, a 5-indazolyl group, a 6-indazolyl group, a 7-indazolyl group, a 1-purinyl group, a 2-purinyl group, a 3-purinyl group, a 6-purinyl group, a 7-purinyl group, a 8-purinyl group, a 2-quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 1-phthalazinyl group, a 5-phthalazinyl group, a 6-phthalazinyl group, a 2-naphthyridinyl group, a 3-naphthyridinyl group, a 4-naphthyridinyl group, a 2-quinoxalinyl group, a 5-quinoxalinyl group, a 6-quinoxalinyl group, a 2-quinazolinyl group, a 4-quinazolinyl group, a 5-quinazolinyl group, a 6-quinazolinyl group, a 7-quinazolinyl group, a 8-quinazolinyl group, a 3-cinnolinyl group, a 4-cinnolinyl group, a 5-cinnolinyl group, a 6-cinnolinyl group, a 7-cinnolinyl group, a 8-cinnolinyl group, a 2-puteridinyl group, a 4-puteridinyl group, a 6-puteridinyl group, a 7-puteridinyl group or a 3-furazanyl group.

The benzyl group which may be substituted by R^{a} may, for example, be a benzyl group, an o-methylbenzyl group, a m-methylbenzyl group, a p-methylbenzyl group, an o-chlorobenzyl group, a m-chlorobenzyl group, a p-chlorobenzyl group, an o-fluorobenzyl group, a p-fluorobenzyl group, an o-methoxybenzyl group, a p-methoxybenzyl group, a p-nitrobenzyl group or a p-cyanobenzyl group.

As the oxoammonium salt to be used in the present invention, one having a skeleton of e.g. 2,2,6,6-tetramethylpiperidine, 2-azaadamantane or [3,3,1]-azabicyclononane may be mentioned as a typical example. Particularly preferred is one having a 2-azaadamantane skeleton, i.e. one represented by the formula (1) wherein R³ and R⁴ together represent CH₂CHR⁷CH₂, provided that each hydrogen atom in CH₂CHR⁷CH₂ may be substituted by R⁵, and R⁶ and R⁷ together form methylene which may be substituted by R⁵. As such an example, an oxoammonium salt of 1-methyl-2-azaadamantane-N-oxyl (hereinafter referred to also as 1-Me-AZADO) or an oxoammonium salt of 2-azaadamantane-N-oxyl (hereinafter referred to also as AZADO) may, for example, be mentioned.

As X⁻, it may be any counter ion, and preferably, for example, F⁻, Cl⁻, Br⁻ or I⁻, and more preferably Cl⁻.

The alkali metal chlorite to be used in the present invention may, for example, be preferably sodium chlorite.

In the present invention, a primary alcohol is oxidized by a catalytic amount of an oxoammonium salt to give a hydroxylamine and an aldehyde. Then, the aldehyde is oxidized to a carboxylic acid by a chlorite, whereby a hypochlorite is produced as a byproduct. It is considered that by this hypochlorite, the hydroxylamine is re-oxidized to an oxoammonium salt thereby to form a catalytic mechanism.

A schematic view of the present invention will be shown in a case where an oxoammonium salt of TEMPO and 1-Me-AZADO disclosed in a literature (Shibuya M. et al., J. Am. Chem. Soc., 2006, vol. 128, p8412) (hereinafter referred to as TEMPO⁺CL⁻ and 1-Me-AZADO⁺Cl⁻, respectively) are used, and as a bulk oxidizing agent, sodium chlorite is used. However, the present invention is by no means limited thereto.

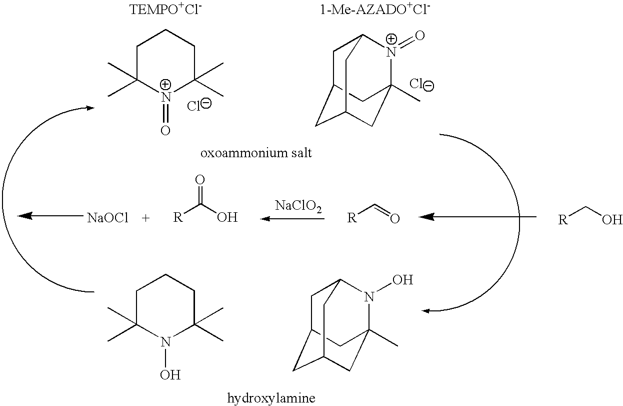

In the present invention, an oxoammonium salt is used as a catalyst, whereby sodium hypochlorite which serves to regenerate the catalyst, will be gently formed in the system, and thus, it is not required to add a catalytic amount of sodium hypochlorite in order to oxidize a nitroxyl radical to an oxoammonium to initiate the reaction, and it is not required to strictly control the reaction.

Further, the hypochlorite is gently formed in the system, whereby it is possible to minimize chlorination of an unsaturated bond or an electron-rich aromatic ring, as a common side reaction.

The oxoammonium chloride may be prepared by reacting the corresponding nitroxyl radical form or hydroxyamine form with chlorine, or by reacting such a precursor with sodium hypochlorite in a solvent, to form it in the reaction system.

For example, in a case where 1-Me-AZADO⁺Cl⁻ is used as a catalyst, an oxidation reaction will proceed smoothly even at room temperature.

In the present invention, the oxidation reaction can be carried out in one-pot by vigorous stirring at room temperature in a mixed solvent of an organic solvent and an acidic buffer solution, containing a various primary alcohol as a substrate, a catalytic amount of an oxoammonium salt, and sodium chlorite as a bulk oxidizing agent.

The solvent is not particularly limited so long as it is one not to hinder the progress of the reaction. For example, dichloromethane is preferred.

As the buffer solution, for example, an aqueous sodium dihydrogen phosphate solution is preferred.

After confirming formation of the carboxylic acid and disappearance of the starting material primary alcohol and its aldehyde product, the desired carboxylic acid may be isolated by a usual purification operation such as distillation of the solvent, extraction, recrystallization, filtration, decantation or column chromatography.

Further, in a case where the progress of the reaction is slow, a phase-transfer catalyst may be added to accelerate the reaction.

Now, the present invention will be described in further detail with reference to Examples, but it should be understood that the present invention is by no means restricted by such Examples.

### Example 1

### Preparation of Oxoammonium Salt

Into a solution of 1-Me-AZADO (1920 mg, 11.55 mmol) in CCl₄ (23.1 mL, 0.5 M), chlorine gas was brown at room temperature, followed by vigorous stirring. Precipitated crystals were collected by a glass filter, washed with cooled Et₂O and dried under reduced pressure to obtain 1-Me-AZADO⁺Cl⁻ (2316 mg, 99%).

### Example 2

### Preparation 2 of Oxoammonium Salt

Using bromine, in the same manner as in Example 1, 1-Me-AZADO⁺Br⁻ (actual counter ion being Br₃⁻) was obtained.

### Example 3

### Oxidation of 3-Phenylpropanol

NaClO₂ (498 mg, 5.507 mmol) was added to a mixed solution containing 150 mg (1.101 mmol) of 3-phenylpropanol in a CH₂Cl₂ (3.7 mL)-NaH₂PO₄ aqueous solution (2.1 mL, 0.52 M solution being 1.0 equivalent), followed by stirring. Then, Me-AZADO⁺Cl⁻ (11.1 mg, 0.05507 mmol) was immediately added, followed by vigorous stirring at room temperature until disappearance of the starting material 3-phenylpropanol and its aldehyde product 3-phenylpropanal was confirmed. After completion of the reaction, 2-methyl-2-butene (1.17 mL, 11.01 mmol) was added under cooling with ice, and an aqueous layer and an organic layer were separated under a weakly acidic condition. To the organic layer, a 10% sodium hydroxide aqueous solution was added to obtain a solution having a pH 11, from which organic substances other than the ionic carboxylic acid were extracted with diethyl ether. The remained aqueous layer was adjusted to pH 3 with 10% hydrochloric acid, and from the aqueous layer, a molecular type carboxylic acid was extracted with diethyl ether. The organic layer was washed with an aqueous sodium chloride solution and then dried over magnesium sulfate, and the solvent was distilled off under reduced pressure.

The residue was dissolved in methylene chloride, and diazomethane was added under cooling with ice, and after confirming the completion of a methyl esterification reaction, stirring was continued at room temperature for a while. Then, the solvent was distilled off under reduced pressure, and the residue was purified by silica gel column chromatography to obtain 176 mg (yield: 97%) of a methyl ester.

Cases wherein the reaction was carried out in the same manner, are shown with respect to the case where Me-AZADO⁺Cl⁻ was employed and the case where TEMPO⁺CL⁻ was employed. Here, in the Table, “alcohol” represents the alcohol, “time (hr)” represents the reaction time (unit: hr), “yield (%)” represents the yield (%) which was calculated by the isolated yield of the methyl ester by the diazomethane. “note” represents a note, “trace” represents a trace amount, “Additive” represents an additive, “SASS” represents sodium stearate, and “slight chlorination” means that slight chlorination was observed. “Cat.” represents the catalyst.

TABLE 1

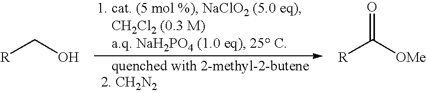

| | | | yield % | | |
|---|---|---|---|---|---|
| No. | alcohol | time [hr] | 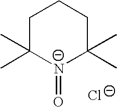 | 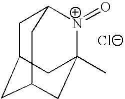 | note |
| 1 | 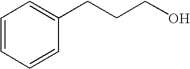 | 1.5 | 77 | 97 | |
| 2 | 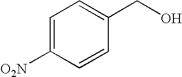 | 1.5 | 63 | 93 | |
| 3 | 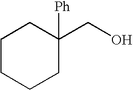 | 10 | trace | 84 | |
| 4 | 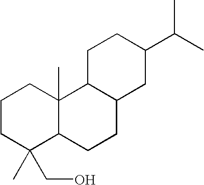 | 32 | trace | 86 | |
| 5 | 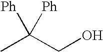 | 5.5/6.5 | trace | 99/96 | Additive (5 mol %) PhCO₂II/ SASS* |
| 6 | 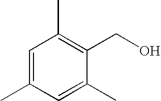 | 4.5 | 20 | 91 | |
| 7 | 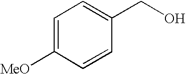 | 10 | 25 | 90 | |
| 8 | 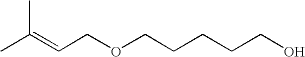 | 24 | <16 | <84 | slight chlorination |
| 9 | 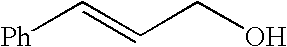 | 3 | <13 | <92 | slight chlorination cat. 20 mol % |
| 10 | 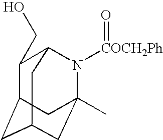 | 5 | — | 92 | |
| 11 | 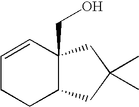 | 1 | — | 88 | |
| 12 | 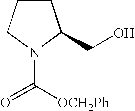 | 15 | trace | 94 | |

SASS*: Stearic Acid Sodium Salt

### INDUSTRIAL APPLICABILITY

According to the present invention, it becomes possible to simply and efficiently produce a desired carboxylic acid not only from a primary alcohol having a relatively simple structure but also from a primary alcohol having various functional groups such as unsaturated bonds, electron-rich aromatic rings, etc., without being chlorinated, and thus, the present invention is very useful for industrial production of pharmaceutical or chemical products.

The entire disclosure of Japanese Patent Application No. 2007-291108 filed on Nov. 8, 2007 including specification, claims and summary is incorporated herein by reference in its entirety.

What is claimed is:

1. A process for producing a carboxylic acid from a primary alcohol, which comprises using an alkali metal chlorite as a co-oxidizing agent and using, as a catalyst, an oxoammonium salt of the formula (1): 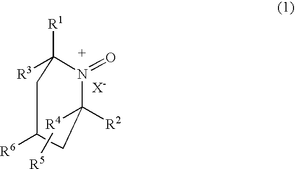
wherein X⁻ is a counter ion, each of R¹, R², R³, R⁴ and R⁵ which are independent of one another, is at least one substituent selected from a hydrogen atom, a halogen atom, a nitro group, a cyano group, a hydroxyl group, a mercapto group, an amino group, a formyl group, a carboxyl group, a sulfo group, a linear or branched C₁₋₁₂ alkyl group, a C₃₋₁₂ cycloalkyl group, a (linear or branched C₁₋₁₂ alkyl)oxy group, a (C₃₋₁₂ cycloalkyl)oxy group, a (linear or branched C₁₋₁₂ alkyl)thio group, a (C³⁻¹² cycloalkyl)thio group, a (linear or branched C₁₋₁₂ alkyl)amino group, a (C₃₋₁₂ cycloalkyl)amino group, a di(linear or branched C₁₋₆ alkyl)amino group, a di(C₃₋₆ cycloalkyl)amino group, a linear or branched C₁₋₁₂ alkylcarbonyl group, a C₃₋₁₂ cycloalkylcarbonyl group, a (linear or branched C₁₋₁₂ alkyl)oxycarbonyl group, a (C₃₋₁₂ cycloalkyl)oxycarbonyl group, a (linear or branched C₁₋₁₂ alkyl)thiocarbonyl group, a (C₃₋₁₂ cycloalkyl)thiocarbonyl group, a (linear or branched C₁₋₁₂ alkyl)aminocarbonyl group, a (C₃₋₁₂ cycloalkyl)aminocarbonyl group, a di(linear or branched C₁₋₆ alkyl)aminocarbonyl group, a di(C₃₋₆ cycloalkyl)aminocarbonyl group, a (linear or branched C₁₋₁₂ alkyl)carbonyloxy group, a (C₃₋₁₂ cycloalkyl)carbonyloxy group, a (linear or branched C₁₋₁₂ alkyl)carbonylthio group, a (C₃₋₁₂ cycloalkyl)carbonylthio group, a (linear or branched C₁₋₁₂ alkyl)carbonylamino group, a (C₃₋₁₂ cycloalkyl)carbonylamino group, a di(linear or branched C₁₋₁₂ alkylcarbonyl)amino group, a di(C₃₋₁₂ cycloalkylcarbonyl)amino group, a linear or branched C₁₋₆ haloalkyl group, a C₃₋₆ halocycloalkyl group, a linear or branched C₂₋₆ alkenyl group, a C₃₋₆ cycloalkenyl group, a linear or branched C₂₋₆ haloalkenyl group, a C₃₋₆ halocycloalkenyl group, a linear or branched C₂₋₆ alkynyl group, a linear or branched C₂₋₆ haloalkynyl group, a benzyl group which may be substituted by R^{a}, a benzyloxy group which may be substituted by R^{a}, a benzylthio group which may be substituted by R^{a}, a benzylamino group which may be substituted by R^{a}, a benzylcarbonyl group which may be substituted by R^{a}, a benzylcarbonyl group which may be substituted by R^{a}, a benzyloxycarbonyl group which may be substituted by R^{a}, a benzylthiocarbonyl group which may be substituted by R^{a}, a benzylaminocarbonyl group which may be substituted by R^{a}, a dibenzylaminocarbonyl group which may be substituted by R^{a}, a benzylcarbonyloxy group which may be substituted by R^{a}, a benzylcarbonylthio group which may be substituted by R^{a}, a benzylcarbonylamino group which may be substituted by R^{a}, a di(benzylcarbonyl)amino group which may be substituted by R^{a}, an aryl group which may be substituted by R^{a}, an aryloxy group which may be substituted by R^{a}, an arylthio group which may be substituted by R^{a}, an arylamino group which may be substituted by R^{a}, a diarylamino group which may be substituted by R^{a}, an arylcarbonyl group which may be substituted by R^{a}, an aryloxycarbonyl group which may be substituted by R^{a}, an arylthiocarbonyl group which may be substituted by R^{a}, an arylaminocarbonyl group which may be substituted by R^{a}, a diarylaminocarbonyl group which may be substituted by R^{a}, an arylcarbonyloxy group which may be substituted by R^{a}, an arylcarbonylthio group which may be substituted by R^{a}, an arylcarbonylamino group which may be substituted by R^{a}, and a di(arylcarbonyl)amino group which may be substituted by R^{a}, provided that when the number of substituents is 2 or more, the respective substituents may be the same or different, R³ and R⁴ may together represent CH₂CHR⁷CH₂, provided that each hydrogen atom in CH₂CHR⁷CH₂ may be substituted by R⁵, each of R⁶ and R⁷ which are independent of each other, has the same meaning as R⁵, or R⁶ and R⁷ may together form methylene which may be substituted by R⁵, R^{a} is halogen, a C₁₋₆ alkyl group, a C₁₋₆ haloalkyl group, a C₃₋₆ cycloalkyl group, a C₁₋₆ alkoxy group, a C₁₋₆ alkoxy C₁₋₆ alkyl group, a C₁₋₆ alkylsulfenyl C₁₋₆ alkyl group, a C₁₋₆ haloalkoxy group, a C₁₋₆ alkylsulfenyl group, a C₁₋₆ alkylsulfinyl group, a C₁₋₆ alkylsulfonyl group, a C₁₋₆ haloalkylsulfenyl group, a C₁₋₆ haloalkylsulfinyl group, a C₁₋₆ haloalkylsulfonyl group, a C₂₋₆ alkenyl group, a C₂₋₆ haloalkenyl group, a C₂₋₆ alkenyloxy group, a C₂₋₆ haloalkenyloxy group, a C₂₋₆ alkenylsulfenyl group, a C₂₋₆ alkenylsulfinyl group, a C₂₋₆ alkenylsulfonyl group, a C₂₋₆ haloalkenylsulfenyl group, a C₂₋₆ haloalkenylsulfinyl group, a C₂₋₆ haloalkenylsulfonyl group, a C₂₋₆ alkynyl group, a C₂₋₆ haloalkynyl group, a C₂₋₆ alkynyloxy group, a C₂₋₆ haloalkynyloxy group, a C₂₋₆ alkynylsulfenyl group, a C₂₋₆ alkynylsulfinyl group, a C₂₋₆ alkynylsulfonyl group, a C₂₋₆ haloalkynylsulfenyl group, a C₂₋₆ haloalkynylsulfinyl group, a C₂₋₆ haloalkynylsulfonyl group, a nitro group, a cyano group, a hydroxyl group, a mercapto group, an amino group, a formyl group, a carboxyl group, a sulfo group, a C₁₋₆ alkoxycarbonyl group, a C₁₋₆ alkylcarbonyl group, a C₁₋₆ haloalkylcarbonyl group, a C₁₋₆ alkylcarbonyloxy group, a phenyl group, a C₁₋₆ alkylamino group or a di-C₁₋₆ alkylamino group, provided that the number of R^{a} is from 1 to 5, and when the number of R^{a} is 2 or more, the respective substituents may be the same or different.

2. The process according to claim 1, wherein in the formula (1), R³ and R⁴ together represent CH₂CHR⁷CH₂, provided that each hydrogen atom in CH₂CHR⁷CH₂ may be substituted by R⁵, and R⁶ and R⁷ together form methylene which may be substituted by R⁵.

3. A process for producing an oxoammonium salt of the formula (3): 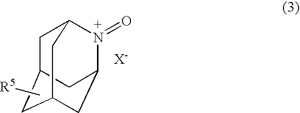
wherein R⁵ and X are as defined above, which comprises reacting halogen with a nitroxyl radical of the formula (2): 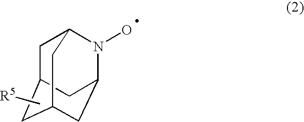
wherein R⁵ is as defined above.

4. The process according to claim 1, wherein the counter ion is F⁻, Cl⁻, Br⁻ or I⁻.

5. The process according to claim 2, wherein the counter ion is F⁻, Cl⁻, Br⁻ or I⁻.

6. The process according to claim 3, wherein the counter ion is F⁻, Cl⁻, Br⁻ or I⁻.
